(19) United States
(12) Patent Application Publication
(10) Pub. No.: US 2002/0187169 A1
(43) Pub. Date: Dec. 12, 2002

(54) Kavalactone compositions

(76) Inventors: Shoujun Chen, Billerica, MA (US); Joel McCleary, The Plains, VA (US); Lijun Sun, Harvard, MA (US)

Correspondence Address: JEFFREY D. HSI, Fish & Richardson P.C., 225 Franklin Street, Boston, MA, 02110-2804, US

(21) Appl. No.: 09/853,304

(22) Filed: May 11, 2001

(51) Int. Cl. A61K 9/70; A61K 31/366

(52) U.S. Cl. 424/401; 514/456

(57) ABSTRACT

This invention relates to compositions having health enhancing qualities, and more particularly to compositions having kavalactones, as well as use and preparation of the compositions.

### BACKGROUND

It is believed that the use of kava (Piper methysticum Forst.) predates written history. The origination of the plant is attributed to the New Guinea/Indonesia area and it is believed that Polynesian explorers were responsible for its spread from island to island. Oceania (i.e., the Pacific island communities of Micronesia, Melanesia and Polynesia) is an area where islanders have been known for centuries to consume a drink, also called kava and derived from the root of kava, in ceremonies and celebrations due to its reported calming effect and ability to promote sociability. The root and the drink were apparently first described in the Western world by Captain James Cook as a result of his exploration of the South Seas in 1768. Many myths and anecdotal stories surround the use of kava, and these vary from culture to culture.

The extract of the kava root is known to contain a class of structurally related chemical compounds known as kavalactones. At least sixteen different members of this chemical class are known to be present. A relaxing action (i.e., calming effect, sleep inducing effect) of the extract is attributed to certain members of this class. Kavalactones possess low bioavailability; in fact, they are practically insoluble in water. Thus, bioavailability in oral administration settings is always an issue that must be addressed. The mechanism of activity of the kavalactones remains uncertain, and their effect on cytokines, such as the interleukins is unclear.

Cytokines such as interleukin-12 (IL-12) mediate the acute phase response to inflammatory stimuli, enhance the microbicidal functions of macrophages and other cells, and promote specific lymphocyte responses. See, e.g., Fearon and Locksley, Science 272:50 (1996). Recently, in vivo studies implicate the inhibition of IL-12 production in therapeutic effects against inflammatory disorders such as sepsis (Zisman et al., Eur. J. Immunol. 27:2994 (1997)), collagen induced arthritis (Malfait et al., Clin. Exp. Immunol. 111:377 (1998)), established colitis (U.S. Pat. No. 5,853,697), experimental autoimmune encephalomyelitis (Leonard et al., J. Exp. Med. 181:381 (1995)), experimental autoimmune uveoretinitis (Yokoi et al., Eur. J. Immunol. 27:641 (1997)), psoriasis (Turka et al., Mol. Med. 1:690 (1995)), and cyclophosphamide induced diabetes (Rothe et al., Diabetologia 40:641 (1997)). Thus, compounds having IL-12 inhibitory activity provide new approaches for therapeutic strategies to address these and other IL-12 mediated disease.

### SUMMARY

The invention is based in part on the unexpected discovery that three kavalactones, dihydrokawain, dihydromethysticin, and kawain (structures shown below), exhibit IL-12 inhibitory activity.

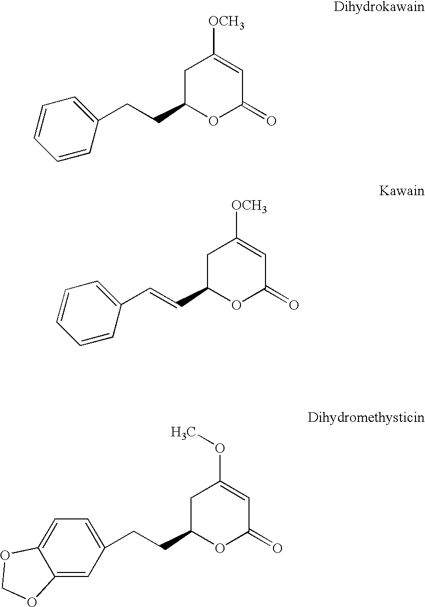

As such, the compounds, compositions and methods of this invention are useful in treating IL-12-mediated disease or disease symptoms (e.g., IL-12 overproduction-related disorders) in a subject. IL-12 mediated disease or disease symptoms refers to disease or disease symptoms in which IL-12 activity is involved, such as those wherein IL-12 is involved in signaling, mediation, modulation, or regulation of the disease process. IL-12 overproduction-related disorders involve those where overproduction of IL-12 is a basis for the disorder.

In one aspect, the invention relates to a medicinal ointment including 1% to 90% (e.g., 1% to 40%, 1.5% to 30%, 2% to 25%) by weight an active kavalactone selected from the group consisting of dihydrokawain, dihydromethysticin, kawain, and a combination thereof, and a medicinally acceptable carrier. The term “active kavalactone” herein refers only to dihydrokawain, dihydromethysticin, kawain, or a combination of them.

In another aspect, the invention is a patch (see, for example, U.S. Pat. No. 5,186,938) including an active kavalactone-containing material layer. More specifically, the material layer, e.g., a pad or a pressure-sensitive adhesive, serves as a substrate for receiving 1% to 90% (e.g., 1% to 40%, 1.5% to 30%, 2% to 25%) by weight an active kavalactone selected from the group consisting of dihydrokawain, dihydromethysticin, kawain, and a combination thereof. The active kavalactone can be in the form of a composition having 1% to 90% (e.g., 1% to 40%, 1.5% to 30%, 2% to 25%) by weight an active kavalactone associated ) with the material layer (e.g., impregnated, embedded, or coated on the surface. A patch optionally has a protective layer intimately adhered to one side of the material layer, which is resistant to passage of the active kavalactone.

The invention also relates to a method for treating (e.g., curing, preventing, or ameliorating) an IL-12 overproduction-related disorder, including administering to a subject (e.g., human, dog, cat) in need thereof an effective amount of an active kavalactone selected from the group consisting of dihydrokawain, dihydromethysticin, kawain, and a combination thereof. The method of treating has an effect on the disease itself or on the symptom. The effect can be objective, that is, a measurable physical effect (e.g., greater range of motion, reduced swelling, reduced rash area), or subjective, that is, based on the feeling or perception of the subject (e.g., decreased irritation, decreased soreness, general feeling of relief). The disorder that can be treated by the method includes colitis, Crohn's disease, diabetes, encephalomyelitis, multiple sclerosis, oesteoarthritis, periodontitis, psoriasis, rheumatoid arthritis, sepsis, and uveoretinitis.

Another aspect of the invention relates to a packaged product including a container, a composition containing an active kavalactone disposed in the container, the kavalactone being selected from the group consisting of dihydrokawain, dihydromethysticin, kawain, and a combination thereof, and a label (e.g., sticker, product insert) with the container and having instructions for application of the active kavalactone for treating an IL-12 overproduction-related disorder.

Also within the invention are a composition herein for use in treating disease (e.g., IL-12 mediated diseases or disease symptoms (such as osteoarthritis), or other diseases (such as fibromylagia), and use of such a composition for the manufacture of a medicament for the treatment of the aforementioned diseases or disease symptoms.

The details of one or more aspects of the invention are set forth in the accompanying FIGURE and the description below. Other features, objects, and advantages of the invention will be apparent from the description and from the claims.

### DESCRIPTION OF DRAWING

FIG. 1 illustrates the IL-12 inhibitory activity of six kavalactones.

### DETAILED DESCRIPTION

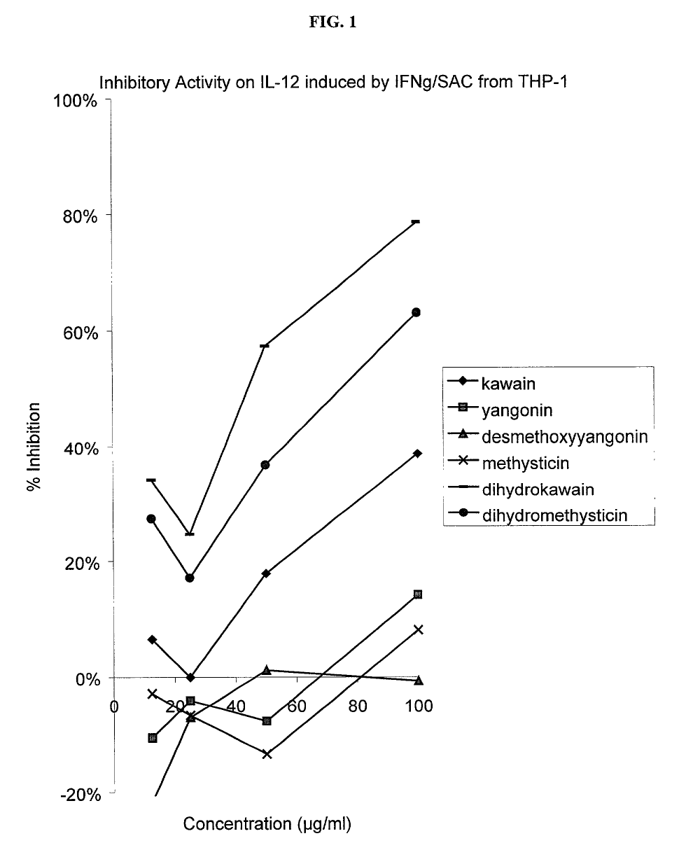

This invention is based in part on the unexpected discovery that specific kavalactones inhibit production of IL-12, whose overproduction is implicated in a number of diseases and disease symptoms. The IL-12 inhibitory activity of six major kavalactones (e.g., desmethoxyyangonin, dihydrokawain, dihydromethysticin, kawain, methysticin, and yangonin) was measured using a cellular assay for determination of IL-12 cytokine inhibition. Among them, kawain, dihydrokawain, and dihydromethysticin were found to have much higher IL-12 inhibitory activity relative to the other kavalactones. These results are shown in FIG. 1. Thus, compositions containing one of the three active kavalactones, kawain, dihydrokawain, dihydromethysticin, or a combination thereof, are useful for treating disease or disease symptoms related to IL-12 overproduction.

Referring back to FIG. 1, six kavalactones were tested in an IL-12 inhibitory assay as follows: Lipopolysaccharide (LPS, Serratia marscencens) was obtained from Sigma (St. Louis, Mo.). Human recombinant IFNg was purchased from Boehringer Mannheim (Mannheim, Germany). Human peripheral blood mononuclear cells were isolated by centrifugation using Ficall-Paque (Pharmacia Biotech, Uppsala, Sweden) and prepared in RPMI medium supplemented with 10% FCS and antibiotics in a 96-well plate with 1×106 cells/well. Human PBMC were primed with IFNγ (30 U/mL) for 16 h and then stimulated with 1 mg/mL of LPS in the presence of different concentrations of test compound. Cell-free supernatants were taken 20 h later for measurement of cytokines. Cell viability was assessed using the bioreduction of MTS [3-(4,5-dimethylthiazol-2-yl)-5-(3-carboxymethoxyphenyl)-2-(4-sulfophenyl)-2H-tetrazolium] (Promega, Madison, Wis.). Cell survival was estimated as the ratio of the absorbance in compound-treated groups versus compound-free control. Human IL-12 was assayed using ELISA kits (Endogen, Cambridge, Mass.), essentially according to the manufacturer's instructions. IL-12 inhibition can also be measured by other methods (e.g., in vivo, in vitro, animal models) of assaying for enzyme inhibition activity.

This invention is also based in part on another unexpected discovery: the active kavalactones, i.e., dihydrokawain, dihydromethysticin, and kawain, can be administered effectively in a transdermal fashion (e.g., as a medicinal ointment). Upon homogeneous formulation in an inert carrier, the active kavalactones can be effectively administered in the absence of permeation enhancers (e.g., dimethyl sulfoxide, 1-dodecyoazacycloheptan-2-one, sodium guaiazulene-3-sulfonate). Thus, compositions of the invention can be administered as an ointment thus avoiding bioavailability problems associated with oral administration (e.g., first pass effects, short half-life in blood, degradation, cytochrome P450 metabolism, gut metabolism, liver or kidney metabolism, or absorption). Such administration techniques allow for systemic or local administration of the dihydrokawain, dihydromethysticin, kawain, or a combination thereof. A medicinal ointment of the invention includes allows for one or more active kavalactones to reach subcutaneous levels, and provides an effect beyond that of a cosmetic or dermapharmaceutical, which affects activities at skin level (e.g., skin cell respiration, regeneration, and hydration).

An ointment composition of the invention can be formulated with one or more of the active kavalactones suspended or dissolved in a carrier, such as mineral oil, liquid petroleum, white petroleum, propylene glycol, polyoxyethylene polyoxypropylene compound, emulsifying wax, water, sorbitan monostearate, polysorbate 60, cetyl esters wax, cetyl alcohol, 2-octyldodecanol, and stearyl alcohol. An acceptable carrier can include water, a solvent, an emollient, a surfactant, a preservative, or a combination thereof. Water, when present, can be in an amount of 5 to 80% by weight. Other than water, the acceptable carrier can also contain a relatively volatile solvent such as a monohydric C1-C3 alkanol (e.g., methyl alcohol or ethyl alcohol) in an amount of 1 to 70% by weight, and an emollient such as those in the form of silicone oils and synthetic esters in an amount of 0.1 to 30% by weight. Other solvents that are acceptable carriers include any suitable for administration of dihydrokawain, dihydromethysticin, and kawain, for example, dimethyl sulfoxide, C1-C20 alcohols, glycols, and ethers. Anionic, nonionic, or cationic surfactants can also be included in the biological acceptable carrier. The concentration of total surfactants can be from 0.1 to 40% by weight. Examples of anionic surfactants include soap, alkyl ether sulfate and sulfonate, alkyl sulfate and sulfonate, alkylbenzene sulfonate, alkyl and dialkyl sulfosuccinate, C8-C20 acyl isethionate, acyl glutamate, C8-C20 alkyl ether phosphate, and a combination thereof. Examples of nonionic surfactants include C10-C20 fatty alcohol or acid hydrophobe condensed with from 2 to 100 moles of ethylene oxide or propylene oxide per mole of hydrophobe; C2-C10 alkyl phenol condensed with from 2 to 20 moles of alkylene oxide; mono and di-fatty acid ester of ethylene glycol; fatty acid monoglyceride; sorbitan, mono- and di-C8-C20 fatty acid; block co-polymer (ethylene oxide/propylene oxide); polyoxyethylene sorbitan, and a combination thereof. Preservatives can also be included in the biological acceptable carrier to prevent growth of potentially harmful microorganisms, and can be employed in an amount of 0.01 to 2% by weight. Examples of preservatives include alkyl ester of para-hydroxybenzoic acid, hydantoin derivative, propionate salt, and a variety of quaternary ammonium compounds. Each preservative should be selected based on its compatibility with other ingredients in the composition. An ointment of this invention can be applied to any particular surface area of the body (including gums).

Also within the scope of the invention is a method for treating an IL-12 overproduction-related disorder, including administering to a subject (e.g., human, dog, cat) in need thereof an effective amount of an active kavalactone selected from the group consisting of dihydrokawain, dihydromethysticin, kawain, and a combination thereof. The effective amount of active kavalactone is between 0.01 and 100 mg/kg body weight per day, alternatively between 0.5 and 75 mg/kg body weight per day of dihydrokawain, dihydromethysticin, kawain, or a combination thereof. The effective amount is useful in a monotherapy or in combination therapy for the treatment of IL-12 overproduction-related disease or disease symptoms. As the skilled artisan will appreciate, lower or higher doses than those recited above may be required. Effective amounts and treatment regimens for any particular subject (e.g., human, dog, cat) will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician or veterinarian.

To practice the method of the present invention, an active kavalactone-containing composition can be administered orally, parenterally, by inhalation spray, topically, rectally, nasally, buccally, vaginally, perineurally, epidurally, by iontophoresis, or via an implanted reservoir. The term “parenteral” as used herein includes subcutaneous, intracutaneous, intravenous, intramuscular, intraarticular, intraarterial, intrasynovial, intrasternal, intrathecal, intralesional and intracranial injection or infusion techniques.

A sterile injectable preparation, for example, a sterile injectable aqueous or oleaginous suspension, can be formulated according to techniques known in the art using suitable dispersing or wetting agents (such as, for example, Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purposes of formulation.

A preparation for oral administration can be any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added. A nasal aerosol or inhalation composition can be prepared according to techniques well-known in the art of pharmaceutical formulation and can be prepared as solutions in saline, employing benzyl alcohol or other suitable preservatives, absorption promoters to enhance bioavailability, fluorocarbons, and/or other solubilizing or dispersing agents known in the art. An active kavalactone-containing composition can also be administered in the form of a suppository or an implantable device. Implantable devices and related technology are known in the art and are useful as delivery systems where a continuous, or timed-release delivery of pure kavalactone compounds or compositions delineated herein is desired. Additionally, the implantable device delivery system is useful for targeting specific points of pure kavalactone compound or composition delivery (e.g., localized sites, or organs). See, Negrin C M, Delgado A, Llabres M and Evora C., Biomaterials 22 (6), 563 (2001). Timed-release technology involving alternate delivery methods can also be used in this invention. For example, timed-release formulations based on polymer technologies, sustained-release techniques and encapsulation techniques (e.g., polymeric, or liposomal) can also be used for delivery of the pure kavalactone compounds and compositions delineated herein. Topical-patches having pure dihydrokawain, dihydromethysticin, kawain or a combination thereof, or a composition thereof are also included in this invention.

Acceptable carriers that can be used to prepare active kavalactone-containing compositions of this invention include, but are not limited to, ion exchangers, alumina, aluminum stearate, lecithin, self-emulsifying drug delivery systems (such as d-α-tocopherol polyethyleneglycol 1000 succinate), surfactants used in pharmaceutical dosage forms (such as Tweens or other similar polymeric delivery matrices), buffer substances (such as phosphates), glycine, sorbic acid, potassium sorbate, partial glyceride mixtures of saturated vegetable fatty acids, water, salts or electrolytes (such as protamine sulfate), disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Other solubilizing agents can also be advantageously used to enhance delivery of dihydrokawain, dihydromethysticin, kawain, or a combination thereof.

Also within the invention is a patch to deliver active kavalactone. A patch includes a material layer (e.g., polymeric, cloth, gauze, bandage) and 1% to 90% (e.g., 1% to 40%) by weight an active kavalactone selected from the group consisting of dihydrokawain, dihydromethysticin, kawain, and a combination thereof. One side of the material layer can have a protective layer adhered to it to resist passage of active kavalactone compositions. The patch can additionally include an adhesive to hold the patch in place on a subject. An adhesive is a composition, including those of either natural or synthetic origin, that when contacted with the skin of a subject, temporarily adheres to the skin. It can be water resistant. The adhesive can be placed on the patch to hold it in contact with the skin of the subject for an extended period of time. The adhesive can be made of a tackiness, or adhesive strength, such that it holds the device in place subject to incidental contact, however, upon an affirmative act (e.g., ripping, peeling, or other intentional removal) the adhesive gives way to the external pressure placed on the device or the adhesive itself, and allows for breaking of the adhesion contact. The adhesive can be pressure sensitive, that is, it can allow for positioning of the adhesive (and the device to be adhered to the skin) against the skin by the application of pressure (e.g., pushing, rubbing,) on the adhesive or device. Also included are peelable masks that can be formulated by placing the composition as a gel or paste on a protective layer made of a film-forming polymer (e.g., polyvinyl alcohol) and an adhesive promoting polymer (e.g., hydrophobic acrylate or methacrylate polymer, such as Pemulen TR2.RTM. from the B.F. Goodrich Company). Alternatively, a hydrogel composition (see, for example, U.S. Pat. No. 5,961,479 or U.S. Pat. No. 5,306,504) including any one or more of the active kavalactones can be used.

The invention also covers a pharmaceutical composition having a pure active kavalactone selected from the group consisting of dihydrokawain, dihydromethysticin, kawain, or a combination thereof. Such a composition is useful for treating IL-12 mediated disease or disease symptoms, or other diseases (such as fibromylagia). Also within this invention is a method of treating disease or disease symptoms, (including IL-12 mediated disease or disease symptoms) in a subject by administering to the subject a pure kavalactone-containing composition. The subject can be a human or an animal (e.g., dog, cat). The term “pure” refers to a level of 90% or higher. Pure active kavalactone can be derived from natural (e.g., root extract and purification) or synthetic (e.g., synthesis from natural or synthetic materials) means, or a combination thereof.

A crude extract of the kava roots (obtained using various extraction methods (e.g., simple solvent soak, supercritical fluid extraction)) can be used as the source of active kavalactones for the preparation of a composition of this invention. If desired, the active kavalactones can be further purified by column chromatography. They can also be synthesized from readily available starting materials by conventional chemical methods. See, for example, Kostermans, Reclk. Trav. Chim. Pays-Bas., 70, 79 (1951); Klohs et al., J. Org. Chem., 24, 1829 (1959); Spino, et al. Tetrahedron Lett., 37, 6503 (1996), and references cited in each. The active kavalactones present in a composition can be enriched by addition of those kavalactones (from either natural or synthetic sources). The three active kavalactones (e.g., dihydrokawain, dihydromethysticin, and kawain) contain one or more asymmetric centers and thus can occur as racemates and racemic mixtures, single enantiomers, individual diastereomers and diastereomeric mixtures. They can also occur in cis- or trans- or E- or Z-double bond isomeric forms. All such isomeric forms can be tested using IL-12 assays to determine their inhibitory activity.

In order that the invention described herein may be more readily understood, the following examples are set forth. It should be understood that these examples are for illustrative purposes only and are not to be construed as limiting this invention in any manner. All references cited herein are expressly incorporated by reference in their entirety.

### EXAMPLE 1

Kawain is synthesized essentially as follows. N-Bromosuccinimide (1 eq.) is slowly added to a 2.3M solution of ethyl β-methoxycrotonate (1 eq.) in carbon tetrachloride. Upon allowing the reaction to equilibrate, the mixture is heated at reflux for ca. 4 h. The mixture is then cooled (0° C.) and filtered, followed by washing of the precipitate with cold CCl₄. The combined filtrates are concentrated (in vacuo, rotovap) and the residue distilled to give the desired product, ethyl γ-bromo-β-methoxycrotonate, whose identity is confirmed by various means including proton nuclear magnetic resonance spectrometry and mass spectrometry.

A 0.5M solution of ethyl γ-bromo-β-methoxycrotonate (1 eq.) in benzene is poured into a flask containing zinc filings (1.2 eq.). Cinnamic aldehyde (1.2 eq.) is added. Upon gentle warming to initiate the reaction, the mixture is refluxed for ca. 1 hr. The mixture is cooled, poured into cooled saturated aqueous ammonium chloride, and the aqueous phase extracted three times with ethyl ether. The combined extracts are dried over sodium sulfate, filtered and concentrated in vacuo. The resulting residue is recrystallized (MeOH) to give to give the desired product whose identity is confirmed by various means including proton nuclear magnetic resonance spectrometry and mass spectrometry.

### EXAMPLE 2

Dihydrokawain is synthesized essentially as follows. Methyl 3-hydroxy-5-phenylpentanoate (1 eq.) in tetrahydrofuran is added to a solution of the lithium enolate of t-butyl acetate (3 eq., from lithium diisopropylamine and t-butyl acetate) at −78° C. and allowed to slowly warm to 0° C. The mixture is quenched with 1N HCl solution and extracted with dichloromethane. The combined extracts are washed with aqueous sodium bicarbonate, brine, dried over sodium sulfate, filtered and concentrated in vacuo to give a residue. The residue can be purified (silica gel chromatography) or converted directly. The resulting β-diketone is hydrolyzed with subsequent lactonization essentially according to the procedure of Tabuchi et al. (trifluoroacetic acid, dichloromethane; J. Org. Chem. 59, 4749, (1994)) to give the desired product, whose identity is confirmed by various means including proton nuclear magnetic resonance spectrometry and mass spectrometry.

### EXAMPLE 3

Dihydromethysticin is synthesized essentially as follows. 10% Palladium on carbon (0.03 wt. eq.) is added to a 1M solution of methysticin (1 eq.) in tetrahydrofuran. The mixture is subjected to hydrogenation using a Parr apparatus at ca. 35 p.s.i. The mixture is filtered and the combined filtrates are concentrated (in vacuo, rotovap) to give a solid. The solid material is recrystallized (ⁱPrOH) to give the desired product, whose identity is confirmed by various means including proton nuclear magnetic resonance spectrometry and mass spectrometry.

### EXAMPLE 4

A crude EtOH extract of kava-kava (100 g) containing about 40 g of kavalactones (PureWorld botanicals, NJ) was suspended into a mixture of water (300 mL) and ethyl acetate (200 mL). After removal of insoluble residues, the organic layer was separated from the aqueous layer. The aqueous layer was further extracted with ethyl acetate (200 mL×2) to produce organic extracts. All organic extracts were combined to obtain an organic solution, which was washed with a saturated NaCl solution (200 mL×2), dried over anhydrous NaSO₄, and dried. The resulting dark brown oil (45 g) was purified by column chromatography with 800 g of Kieselgel 60 (230-400 mesh ASTM, EM Science, Germany), n-hexane/ethyl acetate (2:1) being the eluting solvent. Pale yellow kavalactone fractions were collected and dried to produce a partially crystallized amorphous oil (36 g). The total content of the kavalactones in the product thus obtained was about 93% by weight. Each of the three kavalactones, dihydrokawain, dihydromethysticin, and kawain, was identified by high pressure liquid chromatography.

### EXAMPLE 5

A crude EtOH extract of kava-kava (100 mL) containing about 15 g of kavalactones (PureWorld botanicals, NJ) was concentrated under reduced pressure to remove excess EtOH. The concentrated extract (60 mL) was purified by column chromatography with 500 g of Florisil (200mesh, Aldrich), n-hexane/ethyl acetate (2:1) being the eluting solvent. Yellow kavalactone fractions were collected and dried to produce a pale yellow amorphous oil (13 g). The total content of the kavalactones in the product thus obtained was about 95% by weight.

### EXAMPLE 6

A light yellow kava-kava extract (10 g) containing about 5 g of kavalactones (extracted by Phasex Corp., MA) obtained by a supercritical fluid extraction method (V.J. Krukonis, ACS Symposium Series 289 (1984), pp 155-175) was purified by column chromatography with 300 g Aluminum Oxide, Neutral (J. T. Barker, NJ), with n-hexane/ethyl acetate (2:1) being the eluting solvent. Pale yellow kavalactone fractions were collected and dried to produce a partially crystallized amorphous oil (4.2 g). The total content of the kavalactones in the product thus obtained was about 95% by weight.

### EXAMPLE 7

Composition of a kavalactones-containing cream of this invention:

| chemical name | wt. % |
|---|---|
| kavalactones | 10 |
| glycerin | 1 |
| propylene glycol | 1 |
| polyglycerylmethacrylate | 1 |
| hydroxyethylcellulose | 0.5 |
| magnesium aluminum silicate | 0.5 |
| imidazolidinyl urea | 0.5 |
| disodium EDTA | 0.05 |
| petrolatum | 2 |
| isopropyl palmitate | 5 |
| dimethicone | 0.5 |
| cetyl alcohol | 0.5 |
| isostearic acid | 3 |
| PEG-40 stearate | 1 |
| PEG-100 stearate | 1 |
| sorbitan stearate | 1 |
| glycolic acid | 7 |
| ammonium hydroxide | pH adjusted to 4.4 |
| deionized water | qs to 100% |

### EXAMPLE 8

Composition of another kavalactones-containing cream of this invention:

| chemical name | wt. % |
|---|---|
| kavalactones | 10 |
| Isostearyl Isononanoate | 2.5 |
| propylene glycol | 1 |
| hydroxyethylcellulose | 0.5 |
| magnesium aluminum silicate | 0.75 |
| cocoa butter | 1.2 |
| petrolatum | 2 |
| isopropyl palmitate | 5 |
| dimethicone | 0.5 |
| stearic acid | 3 |
| isostearic acid | 1.5 |
| glycerol stearate | 1.5 |
| PEG-40 stearate | 1 |
| PEG-100 stearate | 1 |
| cetyl/stearyl alcohol | 2.5 |
| glycerin | 2.5 |
| glycolic acid | 10 |
| propylparaben | 0.1 |
| ammonium hydroxide | pH adjusted to 3.8 |
| deionized water | qs to 100% |

### EXAMPLE 9

Composition of another kavalactones-containing cream of this invention:

| chemical name | wt. % |
|---|---|
| beeswax | 24.5 |
| kavalactones | 5 |
| vegetable oil (jojoba oil) | 70 |
| propylparaben | 0.5 |

### EXAMPLE 10

Composition of a cream, to which various amounts of kavalactones can be added:

| ingredient | wt (%) |
|---|---|
| petrolatum | 2 |
| stearyl alcohol | 0.5 |
| isopropyl myristate | 5 |
| sorbitan monooleate | 5 |
| polyoxyl 40 stearate | 5 |
| propylene glycol | 5 |
| methylparaben | 0.3 |
| ammonium hydroxide | pH adjusted to 4.4 |
| deionized water | qs to 100% |

### EXAMPLE 11

Composition of a kavalactones-containing jelly of this invention:

| chemical name | wt. % |
|---|---|
| white petrolatum, USP | 90 |
| kavalactones | 10 |

### EXAMPLE 12

Composition of an oil-in-water emulsion, to which various amounts of kavalactones can be added:

| chemical name | wt. % |
|---|---|
| xanthan gum | 0.2 |
| disodium EDTA | 0.1 |
| sodium PCA | 0.5 |
| diazodinyl urea | 0.3 |
| titanium dioxide | 1 |
| stearic acid | 3 |
| cyclomethicone | 0.3 |
| cetyl alcohol | 0.5 |
| glyceryl stearate | 0.5 |
| PEG-100 stearate | 0.5 |
| steareth-2 | 0.2 |
| lecithin | 0.5 |
| tocopherol | 0.2 |
| octyl methoxycinnamate | 6 |
| glucono-1,5-lactone | 6 |
| glycolic acid | 3 |
| malic acid | 2 |
| lactic acid | 2 |
| green tea extract | 1 |
| triethanolamine | pH adjusted to 3.8 |
| deionized water | qs to 100% |

### EXAMPLE 13

A patient with rheumatoid arthritis (left leg, joint) was unresponsive to several oral medications. A composition containing 5 g of cream (as described in Example 10) and 500 mg of kavalactones (as extract prepared according to Example 4) was administrated to the joint three times a day. Substantial relief of the rheumatoid arthritis symptoms was achieved 30 min after topically applying the kavalactones-containing cream to the joint.

### EXAMPLE 14

A patient suffered from chronic lower back problems, which could not be relieved by oral drugs (such as aspirin and ibuprofen). Substantial relief of the symptoms (e.g., relief from burning sensation in the affected area, general relief to resume daily activity (e.g., walking) was achieved 10 min after applying the kavalactones-containing cream described in Example 13 to the back.

### EXAMPLE 15

A patient suffers from fibromylagia symptoms in the left knee. Ten minutes after applying the kavalactones-containing cream described in Example 13 to the knee, the patient felt relief from discomfort.

### EXAMPLE 16

A patient suffers from periodontitis (molars). Ten minutes after applying a kavalactones-containing jelly described in Example 11 (using kavalactone extract prepared according to Example 4) to the gum area, the symptoms were ameliorated, including reduced redness of the affected area and relief from discomfort.

### Other Embodiments

While a number of embodiments of this invention have been described, it is apparent that they can be altered to provide other embodiments that utilize the products and processes of this invention. Therefore, it will be appreciated that the scope of this invention is to be defined by the claims rather than by the specific embodiments that have been represented by way of example. Accordingly, other embodiments are within the scope of the following claims.

What is claimed is:

1. A medicinal ointment comprising 1% to 90% by weight an active kavalactone selected from the group consisting of dihydrokawain, dihydromethysticin, kawain, and a combination thereof; and a medicinally acceptable carrier.

2. The medicinal ointment of claim 1, wherein the active kavalactone is dihydrokawain.

3. The medicinal ointment of claim 1, wherein the active kavalactone is dihydromethysticin.

4. The medicinal ointment of claim 1, wherein the active kavalactone is a combination of dihydromethysticin, dihydrokawain, and kawain.

5. The medicinal ointment of claim 1, wherein the ointment comprises 1.5% to 30% by weight an active kavalactone selected from the group consisting of dihydrokawain, dihydromethysticin, kawain, and a combination thereof; and a medicinally acceptable carrier.

6. The medicinal ointment of claim 5, wherein the active kavalactone is dihydrokawain.

7. The medicinal ointment of claim 5, wherein the active kavalactone is dihydromethysticin.

8. The medicinal ointment of claim 5, wherein the active kavalactone is a combination of dihydromethysticin, dihydrokawain, and kawain.

9. The medicinal ointment of claim 1, wherein the ointment comprises 2% to 25% by weight an active kavalactone selected from the group consisting of dihydrokawain, dihydromethysticin, kawain, and a combination thereof; and a medicinally acceptable carrier.

10. The medicinal ointment of claim 9, wherein the active kavalactone is dihydrokawain.

11. The medicinal ointment of claim 9 wherein the active kavalactone is dihydromethysticin.

12. The medicinal ointment of claim 9, wherein the active kavalactone is a combination of dihydromethysticin, dihydrokawain, and kawain.

13. A patch comprising a material layer and a composition associated with the material layer; the composition having 1% to 90% by weight an active kavalactone selected from the group consisting of dihydrokawain, dihydromethysticin, kawain, and a combination thereof, associated with the material layer.

14. The patch of claim 13, further comprising a protective layer intimately adhered to one side of the material layer which is resistant to passage of the active kavalactone.

15. The patch of claim 14, wherein the material layer includes a pressure-sensitive adhesive.

16. The patch of claim 15, wherein the active kavalactone is dihydrokawain.

17. The patch of claim 15, wherein the active kavalactone is dihydromethysticin.

18. The patch of claim 15, wherein the active kavalactone is a combination of dihydromethysticin, dihydrokawain, and kawain.

19. The patch of claim 15, wherein the adhesive layer includes 1.5% to 30% by weight an active kavalactone selected from the group consisting of dihydrokawain, dihydromethysticin, kawain, and a combination thereof; and a medicinally acceptable carrier.

20. The patch of claim 19, wherein the active kavalactone is dihydrokawain.

21. The patch of claim 19, wherein the active kavalactone is dihydromethysticin.

22. The patch of claim 19, wherein the active kavalactone is a combination of dihydromethysticin, dihydrokawain, and kawain.

23. The patch of claim 15, wherein the adhesive layer includes 2% to 25% by weight an active kavalactone selected from the group consisting of dihydrokawain, dihydromethysticin, kawain, and a combination thereof; and a medicinally acceptable carrier.

24. The patch of claim 23, wherein the active kavalactone is dihydrokawain.

25. The patch of claim 23, wherein the active kavalactone is dihydromethysticin.

26. The patch of claim 23, wherein the active kavalactone is a combination of dihydromethysticin, dihydrokawain, and kawain.
